(19) United States
(12) Patent Application Publication
(10) Pub. No.: US 2004/0065324 A1
(43) Pub. Date: Apr. 8, 2004

(54) Thermal inhaler

(76) Inventor: Daniel Kozak Pivinski, Voorhees, NJ (US)

Correspondence Address: DANIEL KOZAK PIVINSKI, 6222 MAIN STREET, VOORHEES, NJ, 08043, US

(21) Appl. No.: 10/266,382

(22) Filed: Oct. 8, 2002

(51) Int. Cl. A61M 11/00; A61M 16/00

(52) U.S. Cl. 128/200.23; 128/200.14; 128/203.26

(57) ABSTRACT

An inhaler which provides a controlled delivery of warm air and an inhalant or a warmed inhalant for use by an individual to inhale a particular medicament from a reservoir comprising a Metered-dose, Dry-powder, or Nebulizing inhaler; a heating mechanism and power source; a chamber for housing a heating mechanism and power source; a heating-element trigger; and a conduit defining an air flow path; and

Methods for using variations of said inhaler to assist users in withdrawing from nicotine dependency and/or assisting users in replacing their preferred nicotine application (i.e. Cigarettes, Pipes, Cigars) with a safer and more effective delivery system, which introduces a warmed and possibly flavored predetermined dose of a nicotine medicament into the lungs of the individual; and

An inhaler which provides a controlled delivery of warm air into a user's respiratory tract; and

A method of quickly and comfortably increasing a user's temperature by warming them from the inside by introducing warm air into their respiratory tract.

### BACKGROUND

1. Field of the Invention

This invention relates to an inhaler.

2. Description of Related Art

There are currently three main methods for drug delivery via the respiratory tract, namely metered dose inhalers, dry powder inhalers, and nebulizers.

Metered dose inhalers (“MDI”) are widely used in the management of asthma. The MDI comprises a drug packaged with a propellant in a pressurized aerosol container can having a valve which releases a volumetric metered dose of aerosol upon actuation. These inhalers are portable, small, and convenient to carry but deliver a dose which varies in quantity, delivery speed, and droplet size distribution as the vapor pressure of the propellant varies. The propellant pressure varies with temperature and decreases progressively as the content becomes depleted so that the range in dose variation may be substantial. Incomplete evaporation of the propellant may cause “sticking” and localized concentration of drug droplets at an impact area, and this in turn can cause undesirable side effects. For example bronchosteroids can cause local immuno-suppression and local fungal infection while local concentration of bronchodilator can lead to swallowing, with unwanted systemic affects. In addition, the use of an MDI requires a degree of synchronization between manual valve actuation and inhalation which many users find difficult.

Dry powder inhalers (“DPI”) devices rely upon a burst of inspired air to fluidize and draw a dose of an active powder into the bronchial tract. While this avoids the synchronization problem of the MDI, DPI's are sensitive to humidity and may provoke asthma attacks in some individuals sensitive to inhaled powder. Moreover, because the force of inspiration varies from person to person, the dose administered varies.

Nebulizers generate an aerosol by atomizing a liquid in a carrier gas stream and require a continuous gas compressor or bulky supply of compressed gas. In general, the droplet size of the aerosol is a function of carrier gas pressure and velocity and hence cannot be easily varied independently of concentration of the active substance in the gas stream. Inhalation reduces the pressure at the nebulizer nozzle and thus dosage and particle size are also influenced by the duration and strength of each breath. Most nebulizers operate continuously during inhalation and exhalation but special control systems can be employed to meter the aerosolized gas flow from the nebulizer to a holding chamber from which the user may draw a charge.

In addition, many attempts have been made to provide a cigarette substitute which provides nicotine by inhalation but which avoids the need for combustion of tobacco. Provision of a cigarette substitute involves complexities additional to those involved in the administration of a therapeutic agent. Although it is relatively easy to administer nicotine (for example in tablet form, via transdermal patches and the like), such forms do not satisfy habitual smokers because they do not satisfy important complex physiological and psychological affinities acquired by habitual smokers of combustible cigarettes.

In an attempt to provide an acceptable alternative, many cigarette substitutes have been proposed which provide nicotine on inhalation without combustion of tobacco. Conceptually, such devices are less harmful to the inhaler than smoking, avoiding the hazards of, passive smoking among bystanders, and the fire hazard and environmental problems associated with cigarette smoking.

Early cigarette substitutes employed a porous carrier impregnated with a liquid nicotine containing composition through which an air stream could be drawn to volatilize nicotine. This approach yielded insufficient nicotine per puff, suffered from a tendency for the carrier to dry out and delivered a variable amount of nicotine per puff, depending on factors such as air temperature, humidity, lung capacity of the user and amount of liquid composition remaining in the carrier.

Subsequent devices delivered nicotine from a pressurized aerosol container from which nicotine can be released by mechanical valve actuator. In one such device the valve is microprocessor controlled to limit the frequency and duration of actuation. However, the dose delivered varies with the vapor pressure of aerosol remaining in the container as well as with duration of valve actuation. The disposable pressure container, aerosol valve, and CFC propellant add considerably to active substance cost. These devices share the disadvantages of MDI devices previously discussed.

In yet other devices a nicotine containing substance is heated to vaporize an amount of nicotine which is then available for inhalation. The amount of nicotine delivered by such devices is difficult to control and is temperature dependant. In one such device a plurality of nicotine-containing pellets may be heated sequentially so that each liberates a predetermined dose. However, in that case, the dose is fixed during pellet manufacture, particle size of the aerosol is uncontrolled, and temperature of the inhaled air cannot be varied independently of dose.

While each of the inhalers and cigarette substitutes proposed thus far adequately perform their intended primary task of delivering medicament to the user's respiratory tract, none of the related proposals to date are primarily concerned with delivering the intended medicament in such as way (i.e. non-combustible, heated) as to maximize both the user's comfort and ability to synthesize the inhalant by incorporating a variable heating mechanism. Factors such as the quantity of nicotine per puff, the temperature of the puff, the draw, the presence and size distribution of flavor particles in the puff and like factors are of considerable importance in satisfying habitual smokers. The various alternatives proposed to date have simply proved unacceptable to most smokers.

To date no device has provided a satisfactory means of specifically modulating the temperature of the resulting medicament mixture. Thusly, the devices have failed to reap the benefits that heat has on respiration, as they relate to the synthesis of a particular medicament within the lungs or to adequately to mimic the sensations obtained during smoking.

Finally, no device exists that specifically intends to increase a user's temperature by warming them from the inside by providing a controlled delivery of warm air into their respiratory tract.

### SUMMARY OF THE PRESENT INVENTION

This invention relates to an inhaler, and more particularly to an inhaler which provides users with increased comfort during use and a more effective synthesis of an intended medicament by incorporating a heating mechanism, which is intended to warm the said medicament mixture immediately prior to inhalation by the user; and a method of assisting a person to withdraw from nicotine dependency and/or assisting a person to replace their preferred nicotine application with a safer and more effective delivery system by using the invention or a variation thereof. This invention further relates to an inhaler which increases a user's temperature and soothes their anxiety by warming them from the inside by providing a controlled delivery of warm air into their respiratory tract.

### OBJECTIVES OF THE INVENTION

An object of the invention is to provide an improved inhaler.

Another object of the invention is to increase the effectiveness of existing drug delivery methods via the respiratory tract by introducing heat as a catalyst for metabolism of particular medicaments.

Yet another object of the invention is to increase the effectiveness of existing gaseous element dispensers by introducing heat as a catalyst for synthesis of particular vapors by the respiratory tract.

Still another object of the invention is to increase the comfort of users of existing inhalers.

Still another object of the invention is to provide a method to assist users in withdrawing from nicotine dependency.

A further object of the invention is to provide an apparatus to assist users in withdrawing from nicotine dependency.

A further object of the invention is to provide a method to assist users in replacing their preferred nicotine application with a safer delivery system.

A further object of the invention is to provide an apparatus to assist users in replacing their preferred nicotine application with a safer delivery system.

A further object of the invention is to provide an apparatus and method to assist users in either replacing their preferred nicotine application with a safer delivery system or to assist users in withdrawing from nicotine dependency with a delivery system that simulates the sensation of smoking combusted tobacco.

A further object of the invention is to provide an apparatus and method to assist users in either replacing their preferred nicotine application with a safer delivery system or to assist users in withdrawing from nicotine dependency with a delivery system that simulates the sensation of smoking combusted tobacco without exposing the user to potentially harmful medicaments (i.e. Nicotine).

A further object of the invention is to provide an apparatus that quickly and comfortably increases a user's temperature by warming them from the inside by introducing heated air into their respiratory tract.

A further object of the invention is to provide a method of quickly and comfortably increasing a user's temperature by warming them from the inside by introducing heated air into their respiratory tract.

A further object of the invention is to provide a device that and method of quickly and comfortably soothing a user's anxiety by taking advantage of the calming effects of warm air introduced within the respiratory tract.

A further object of the invention is to provide an improved, portable Nebulizing inhaler.

### BRIEF DESCRIPTION OF THE FIGURES

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective and sectional view of the inhaler claimed in claim 7 with a pushbutton as the heating mechanism trigger, a battery as a power source, and a coiled heating-element as a heating mechanism;

FIG. 2 is a perspective and sectional view of the inhaler claimed in claim 7 with a breath activated heating mechanism trigger in the form of a one-way air input fan, a self-generating power source, and a coiled heating-element as a heating mechanism;

FIG. 3 is a perspective and sectional view of the inhaler claimed in claim 7 with a breath activated heating mechanism trigger in the form of a one-way air input fan, a battery as a power source, and a coiled heating-element as a heating mechanism;

FIG. 4 is a perspective and sectional view of the inhaler claimed in claim 2 in the form of a modified “Nicotrol Inhaler” with a breath activated heating mechanism trigger in the form of a trigger flaps housed within the mechanism and secured by spring-resetting, one-way hinges, a battery as a power source, and a coiled heating-element as a heating mechanism;

FIG. 5A is a perspective view of the inhaler claimed in claim 3 with a heating mechanism in the form of a warm-air compressor;

FIG. 5B is a perspective and sectional view of the inhaler claimed in claim 3 with a heating mechanism in the form of a coiled heating element;

FIG. 6 is a perspective and sectional view of the inhaler claimed in claim 7 with a breath activated heating mechanism trigger in the form of a trigger flaps housed within the mechanism and secured by spring-resetting, one-way hinges, a battery as a power source, and a coiled heating-element as a heating mechanism;

FIG. 7A is a perspective and sectional view of the inhaler claimed in claim 1 with a bi-level push button that activates the heating mechanism and dispenses the intended medicament, a battery as a power source, and a coiled heating-element as a heating mechanism;

FIG. 7B is a perspective and sectional view of the inhaler claimed in claim 1 with a standard metered-dose inhaler and a battery-powered, push-button activated, attached heating chamber.

### DETAILED DESCRIPTION OF THE FIGURES

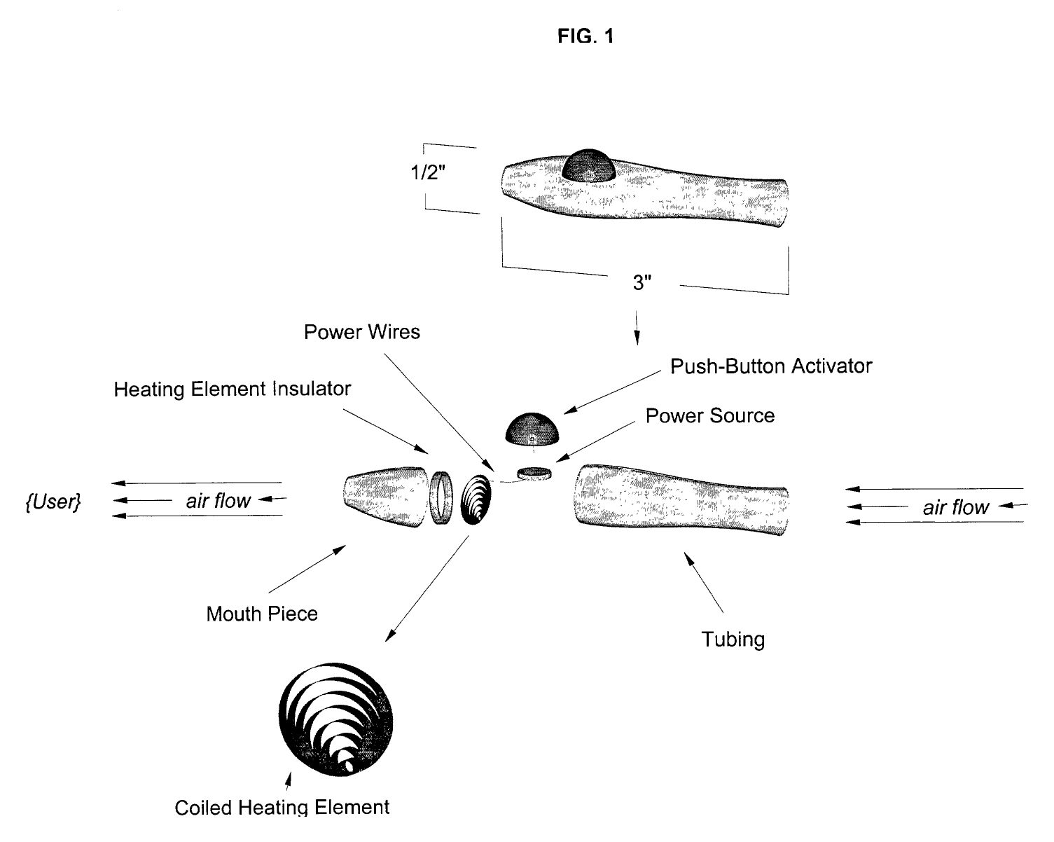

FIG. 1 embodies the invention summarized in claim 7 and consists of a hollow tubular body, a mouth piece, a push-button mechanism, a battery, a coiled heating element which allows inhaled air to pass through itself, connecting wires, and a heating mechanism insulator. The mouth piece portion is intended to be replaceable and exhibits a threaded portion at its connecting end. The body has an accommodating threaded portion at its connecting end. The push-button is connected to the battery and heating element by way of the connecting wires. When a user actuates the device by placing the mouth piece of the assembled device in their mouth, pushing the push-button, and inhaling, the depressed push button shall complete a circuit between the power source and heating element, and air will enter through the end of the device, pass through the activated heating mechanism and thereby be warmed to a specific temperature range, and enter the user's respiratory tract.

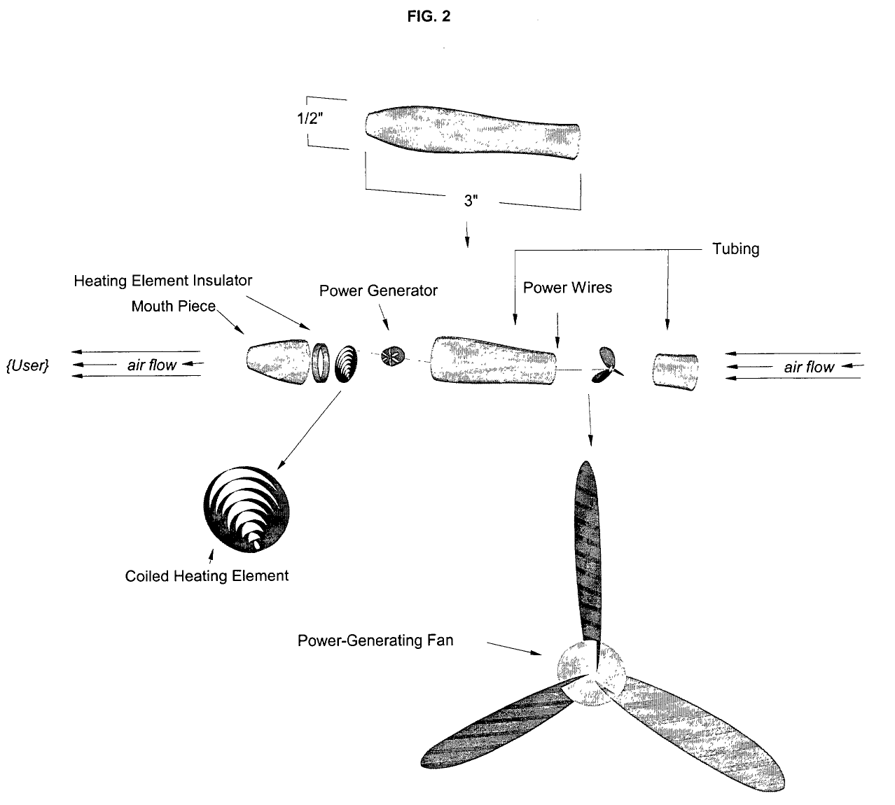

FIG. 2 embodies the invention summarized in claim 7 and consists of a hollow tubular body, a mouth piece, a one-way power generating fan, a power generator, a coiled heating element which allows inhaled air to pass through itself, connecting wires, and a heating mechanism insulator. The mouth piece portion is intended to be replaceable and exhibits a threaded portion at its connecting end. The body has an accommodating threaded portion at its connecting end. The fan is connected to the generator and heating element by way of the connecting wires. When a user actuates the device by placing the mouth piece of the assembled device in their mouth and inhaling, the inhaled air shall pass over the slanted blades of the fan and thereby coerce the fan into motion. The moving fan shall move the components of the generator so as to provide power to the heating mechanism. The fan shall be set to move in one direction only so as to prevent mis-queues of the heating mechanism during user exhale. As the device is actuated, air will enter through the end of the device, proceed through the fan manipulating it into motion, pass through the activated heating mechanism and thereby be warmed to a specific temperature range, and enter the user's respiratory tract.

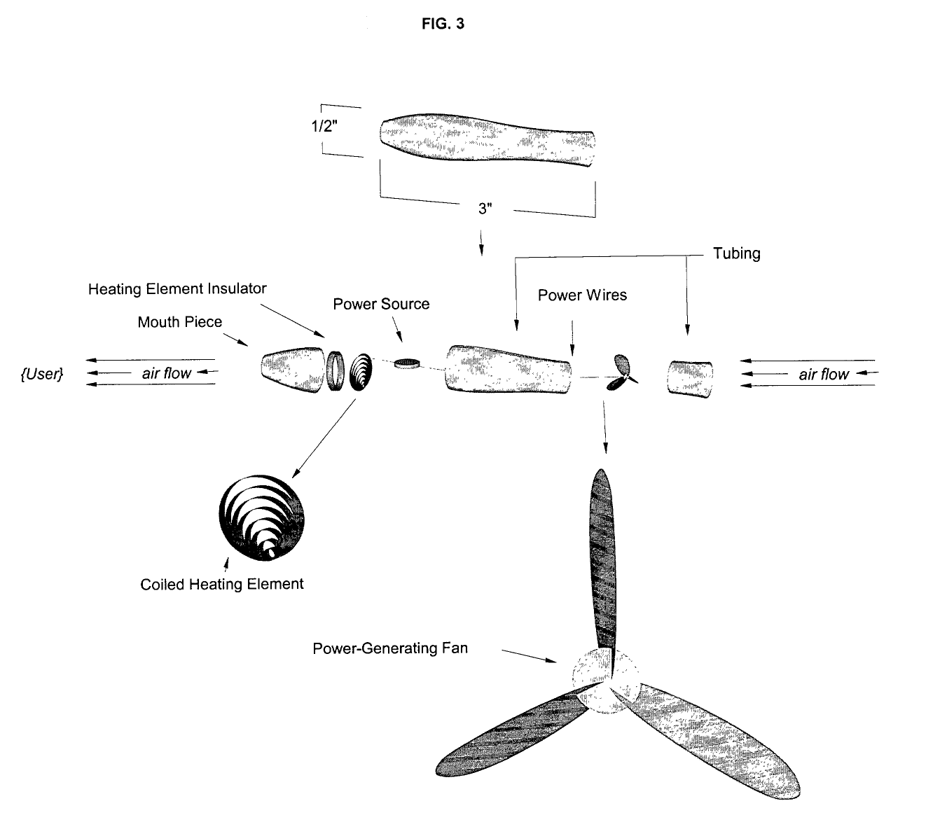

FIG. 3 embodies the invention summarized in claim 7 and consists of a hollow tubular body, a mouth piece, a one-way, heating mechanism-activating fan, a battery, a coiled heating element which allows inhaled air to pass through itself, connecting wires, and a heating mechanism insulator. The mouth piece portion is intended to be replaceable and exhibits a threaded portion at its connecting end. The body has an accommodating threaded portion at its connecting end. The fan is connected to the battery and heating element by way of the connecting wires. When a user actuates the device by placing the mouth piece of the assembled device in their mouth and inhaling, the inhaled air shall pass over the slanted blades of the fan and thereby coerce the fan into motion. The moving fan shall complete a circuit between the power source and heating mechanism by providing contact between the wires power source and thereby activate the heating mechanism. The fan shall be set to move in one direction only so as to prevent mis-queues of the heating mechanism during user exhale and house near its center circuit connectors placed 180 degrees in relation to one another. As the device is actuated, air will enter through the end of the device, proceed through the fan manipulating it into motion, pass through the activated heating mechanism and thereby be warmed to a specific temperature range, and enter the user's respiratory tract.

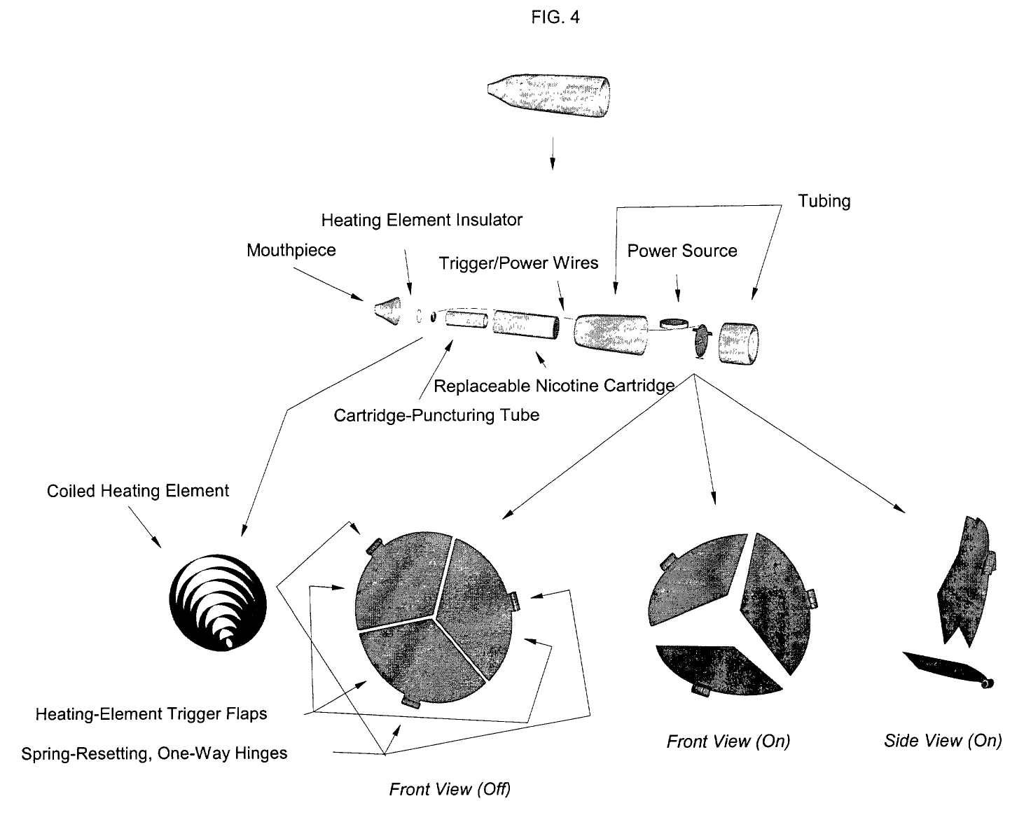

FIG. 4 embodies the invention summarized in claim 2 and consists of a “Nicotrol Inhaler” modified to include heating mechanism-trigger flaps set to the mechanism on spring-resetting one-way hinges, a battery, connecting wires, a coiled heating mechanism, and a heating mechanism insulator. When a user actuates the device by placing the mouth piece of the assembled device in their mouth and inhaling, the resulting vacuum created in the mechanism's main chamber shall pull the trigger flaps open, which will immediately allow air to pass from one side of the invention to the other. Furthermore, when the flaps open, the circuit between the power source and heating mechanism will become complete by providing contact between the wires and trigger flaps, and thereby activate the heating mechanism. The trigger flaps shall be set to move in one direction only so as to prevent mis-queues of the heating mechanism during user exhale. As the device is actuated, air will enter through the end of the device, proceed through the trigger flap section, pass through the medicament cartridge and activated heating mechanism and thereby be warmed to a specific temperature range, and enter the user's respiratory tract.

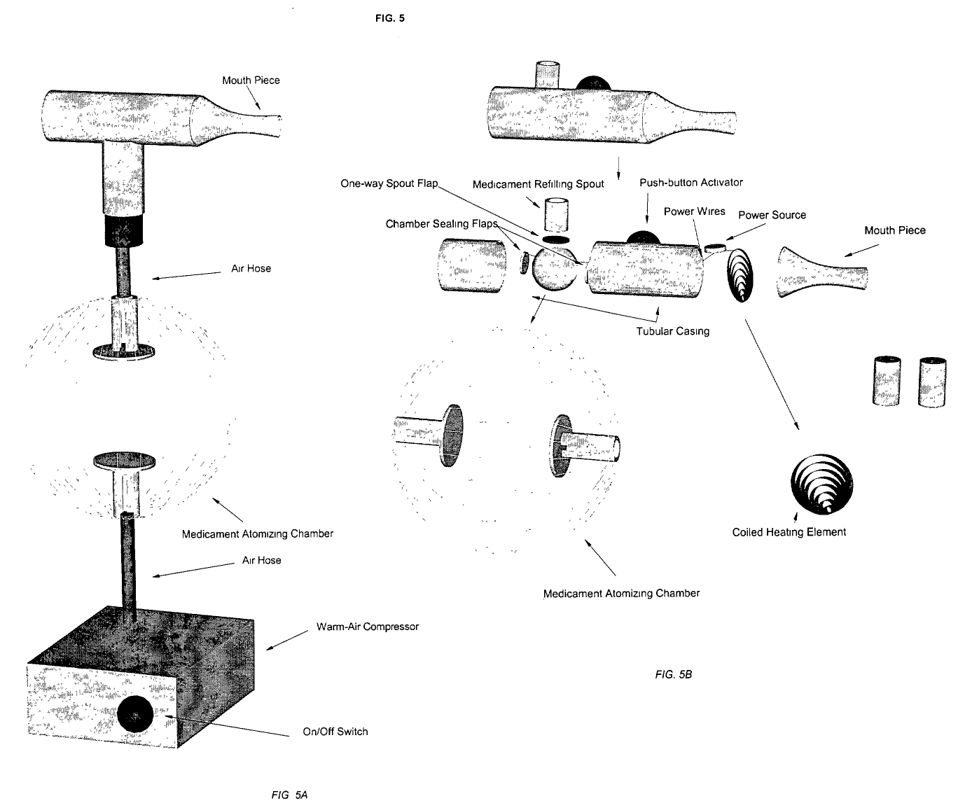

FIG. 5A embodies the invention summarized in claim 3 and consists of a Nebulizing inhaler and a heating mechanism in the form of a warm-air compressor. When a user actuates the device by plugging the compressor into a standard power source, supplying a particular medicament into the atomizing chamber, turning on the compressor, and placing the mouth piece of the assembled device in their mouth and inhaling, warm air will be forced through the chamber thereby creating a medicament-rich aerosol, pass through the delivery mechanism and enter the user's respiratory tract upon inhalation.

FIG. 5B embodies the invention summarized in claim 3 and consists of a new variation of a Nebulizing inhaler and a heating mechanism in the form of a coiled heating element. During filling, the One-Way Spout Flap gives way to allow for the medicament to enter the chamber while disallowing leakage after filling. When a user actuates the device by depressing the Push-button Activator and inhaling, both Chamber Sealing Flaps open thereby allowing free airflow and the heating mechanism activates. Air inhaled by the user into the device enters the chamber, which atomizes the intended medicament, proceeds through and is warmed by the heating and enter the user's respiratory tract through the mouth piece.

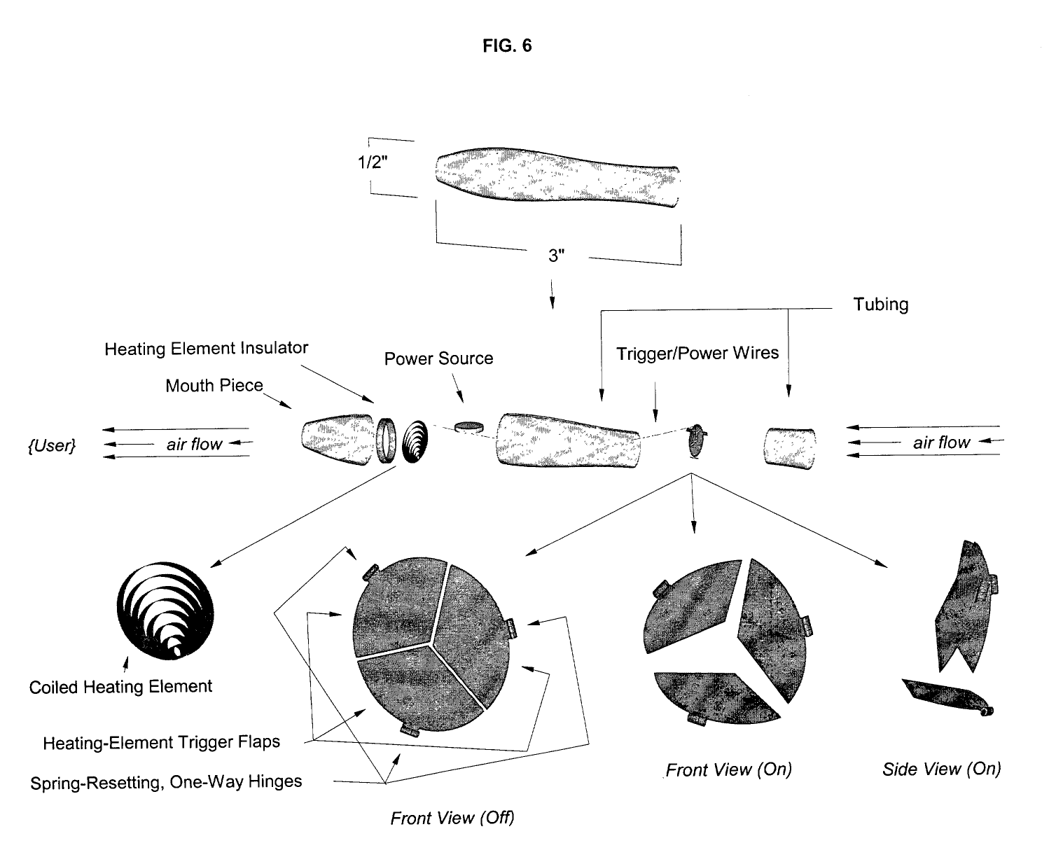

FIG. 6 embodies the invention summarized in claim 7 and consists of a hollow tubular body, a mouth piece, one-way heating mechanism-trigger flaps set to the mechanism on spring-resetting one-way hinges, a battery, a coiled heating element which allows inhaled air to pass through itself, connecting wires, and a heating mechanism insulator. The mouth piece portion is intended to be replaceable and exhibits a threaded portion at its connecting end. The body has an accommodating threaded portion at its connecting end. When a user actuates the device by placing the mouth piece of the assembled device in their mouth and inhaling, the resulting vacuum created in the mechanism's main chamber shall pull the trigger flaps open, which will immediately allow air to pass from one side of the invention to the other. Furthermore, when the flaps open, the circuit between the power source and heating mechanism will become complete by providing contact between the wires and trigger flaps, and thereby activate the heating mechanism. The trigger flaps shall be set to move in one direction only so as to prevent mis-queues of the heating mechanism during user exhale. As the device is actuated, air will enter through the end of the device, proceed through the trigger flap section, pass through the activated heating mechanism and thereby be warmed to a specific temperature range, and enter the user's respiratory tract.

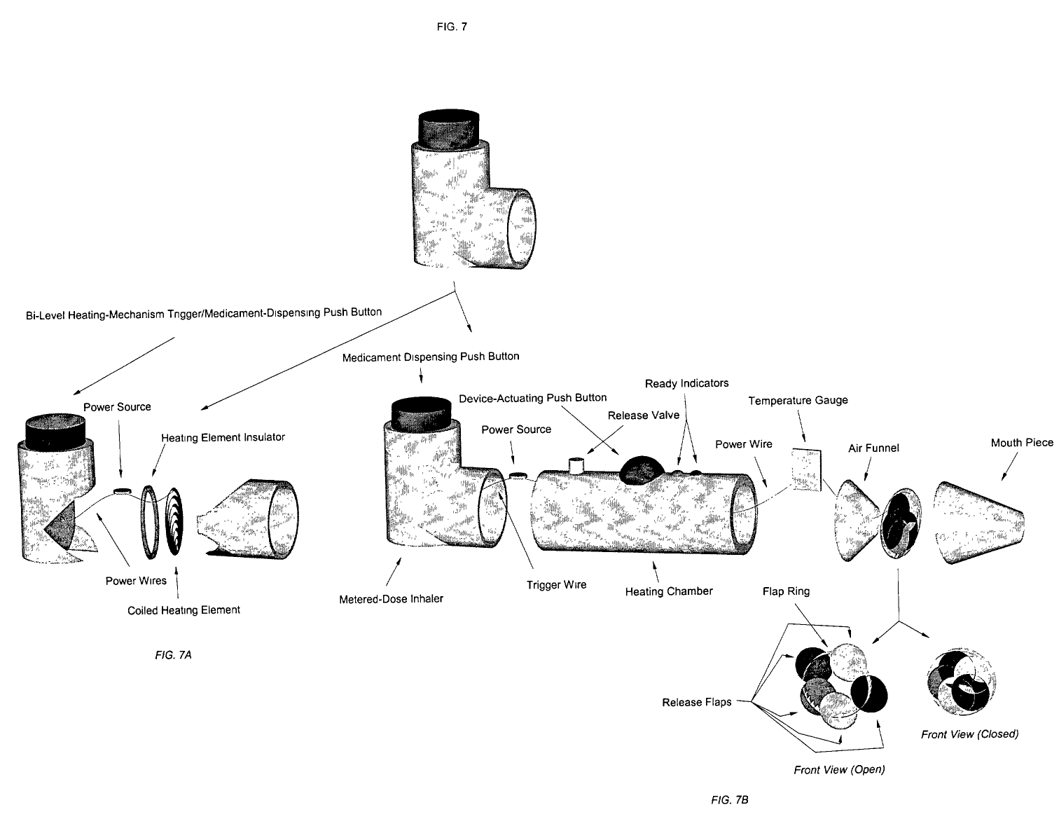

FIG. 7A embodies the invention summarized in claim 1 and consists of a metered-dose inhaler modified to include heating mechanism, a bi-level heating-element trigger/medicament-dispensing button, a battery, connecting wires, a coiled heating mechanism, and a heating mechanism insulator. When a user actuates the device by pushing the button to its first depressed position, the heating mechanism shall activate. The user then fully depresses the button which releases the medicament based aerosol through the device's heating mechanism, out its mouth piece and into the user's respiratory tract.

FIG. 7B embodies the invention summarized in claim 1 and consists of a metered-dose inhaler and heating chamber that is fitted to the inhaler's mouth piece at one end, the chamber comprising a device-actuating push button, a release valve, two ready indicators, a temperature gauge, power wires, release flaps, a release flap ring, an air funnel, infrared heating plates, and a replaceable clip-on mouth piece. When a user actuates the device by pushing the inhaler's button, the heating mechanism in the chamber shall activate and the medicament aerosol shall enter the chamber and be warmed to a specific temperature range. The user then depresses the device actuating button which opens the release valve and release flaps and releases the warmed medicament-based aerosol out the chamber's mouth piece and into the user's respiratory tract upon inhalation.

The components of this embodiment may be replaced with similar constituents, which together serve to perform the same function. For example, the coiled heating element based system may be replaced with an infrared heating plate or resistance element based heating mechanism or the like. Furthermore, any release, power, activating or heating mechanism referenced on any of the embodiments listed above may be used to replace a reciprocal mechanism on any other embodiment.

1. A medicament dispensing device, said device being adapted to release warmed medicament-bearing vapors drawn through said device; said device comprising:
a) a Metered-dose inhaler; and
b) a heating mechanism and power source which will serve to warm the aerosol to be delivered to the patient to a specific temperature that is safe for and comforting to the patient and stimulates a more effective assimilation of medicament by the patient's lungs; and
c) a chamber for housing the heating mechanism and power source which will serve to insulate the heat manifested by the element; and
d) a heating-element trigger to be housed within the device, which is to be set by default to its ‘Off’ position and return to its ‘Off’ position when trigger ceases to be activated, is activated by the patient actuating the device, activates the heating mechanism when triggered, and resets the heating mechanism to its default ‘Off’ state when ceasing to be activated; and
e) a conduit defining an air flow path extending from the pressurized aerosol container, through the heating mechanism, and to the mouth piece; and possibly
f) an elective chamber to house a vapor flavoring; and possibly
g) an elective manual switch which allows the user to modulate the temperature of the resulting inhalant.

2. A medicament dispensing device, said device being adapted to release warmed medicament-bearing vapors compelled through said device; said device comprising:
a) a Dry-powder inhaler; and
b) a heating mechanism and power source which will serve to warm the medicament/air mixture to be delivered to the patient to a specific temperature that is safe for and comforting to the patient and stimulates a more effective assimilation of medicament by the patient's lungs; and
c) a chamber for housing the heating mechanism and power source which will serve to insulate the heat manifested by the element; and
d) a heating-element trigger to be housed within the device, which is to be set by default to its ‘Off’ position and return to its ‘Off’ position when trigger ceases to be activated, is activated by the patient actuating the device, activates the heating mechanism when triggered, and resets the heating mechanism to its default ‘Off’ state when ceasing to be activated; and
e) a conduit defining an air flow path extending from one end of the reservoir, through the heating mechanism, and to the other; and possibly
f) an elective chamber to house a vapor flavoring; and possibly
g) an elective manual switch which allows the user to modulate the temperature of the resulting inhalant.

3. A medicament dispensing device, said device being adapted to release warmed medicament-bearing or other (i.e. oxygen) vapors drawn through said device; said device comprising:
a) a Nebulizing inhaler; and
b) a heating mechanism and power source which will serve to warm the aerosol to be delivered to the patient to a specific temperature that is safe for and comforting to the patient and stimulates a more effective assimilation of medicament by the patient's lungs; and
c) a chamber for housing the heating mechanism and power source which will serve to insulate the heat manifested by the element; and
d) a heating-element trigger to be housed within the device, which is to be set by default to its ‘Off’ position and return to its ‘Off’ position when trigger ceases to be activated, is activated by the patient actuating the device, activates the heating mechanism when triggered, and resets the heating mechanism to its default ‘Off’ state when ceasing to be activated; and
e) a conduit defining an air flow path extending from the compressor, through the heating mechanism, and to the delivery conduit (i.e. mouth piece or nasal fitting); and possibly
f) an elective chamber to house a vapor flavoring; and possibly
g) an elective manual switch which allows the user to modulate the temperature of the resulting inhalant.

4. A method of assisting a person to withdraw from nicotine dependency and/or to replace their preferred nicotine application (i.e. Cigarettes, Pipes, Cigars) by introducing a predetermined dose of a nicotine medicament into the lungs of an individual using the inhaler to which is referred in claim 1; the method comprising:
a. Introducing a warmed, predetermined dose of the particular nicotine-based medicament, which may or may not be flavored, into the air conduct; and
b. Creating an air flow from the pressurized aerosol container, through the heating mechanism and to the device's delivery conduit; and
c. Triggering the heating mechanism to activate when the patient actuates the device; and
d. Triggering the heating mechanism to deactivate when the patient ceases to actuate the device; and possibly
e. Flavoring the medicament mixture by having the resulting aerosol to pass through an elective flavoring chamber, which houses a replaceable aerosol flavoring compound;

5. A method of assisting a person to withdraw from nicotine dependency and/or to replace their preferred nicotine application (i.e. Cigarettes, Pipes, Cigars) by introducing a predetermined dose of a nicotine medicament into the lungs of an individual using the inhaler to which is referred in claim 2; the method comprising:
a. Introducing a warmed, predetermined dose of the particular nicotine-based medicament, which may or may not be flavored, into the air conduct; and
b. Creating an air flow from one end of the reservoir, through the heating mechanism, and to the device's delivery conduit; and
c. Triggering the heating mechanism to activate when the patient actuates the device (i.e. breath activated); and
d. Triggering the heating mechanism to deactivate when the patient ceases to actuate the device; and possibly
e. Flavoring the medicament mixture by having the resulting inhalant to pass through an elective flavoring chamber, which houses a replaceable vapor flavoring compound;

6. A method of assisting a person to withdraw from nicotine dependency and/or to replace their preferred nicotine application (i.e. Cigarettes, Pipes, Cigars) by introducing a predetermined dose of a nicotine medicament into the lungs of an individual using the inhaler to which is referred in claim 3; the method comprising:
a. Introducing a warmed, predetermined dose of the particular nicotine-based medicament, which may or may not be flavored, into the air conduct; and
b. Creating an air flow from the compressor, through the heating mechanism and to the device's delivery conduit; and
c. Triggering the heating mechanism to activate when the device is actuated; and
d. Triggering the heating mechanism to deactivate when the device ceases to be actuated; and possibly
e. Flavoring the medicament mixture by having the resulting aerosol to pass through an elective flavoring chamber, which houses a replaceable aerosol flavoring compound.

7. An inhaler which increases a user's temperature by warming them from the inside by providing a controlled delivery of warm air into their respiratory tract; said device comprising:
a. a heating mechanism and power source which will serve to warm the air to be delivered to the user to a specific temperature that is safe for and comforting to the individual; and
b. a chamber for housing the heating mechanism and power source which will serve to insulate the heat manifested by the element; and
c. a heating-element trigger to be housed within the device, which is to be set by default to its ‘Off’ position and return to its ‘Off’ position when trigger ceases to be activated, is activated by the patient actuating the device, activates the heating mechanism when triggered, and resets the heating mechanism to its default ‘Off’ state when ceasing to be activated; said trigger can be either manual, which would require physical manipulation by user to activate (i.e. a button or pressure chamber), or breath-activated, which would activate upon inhalation through said device by user; and
d. a conduit defining an air flow path extending from one end of the reservoir, through the heating mechanism, and to the other; and
e. a housing that encompasses all components of the invention; and
f. an elective chamber to house a vapor flavoring; and
g. an elective manual switch which allows the user to modulate the temperature of the resulting inhalant.

8. A method of assisting a person to increase their temperature by warming them from the inside by providing a controlled delivery of warm air into their respiratory tract; the method comprising:
a. use of a Metered-dose Inhaler, Dry-powder Inhaler, Nebulizing Inhaler, or the inhaler Claimed in claim 7 of this invention to increase an individual's temperature; and
b. introducing a warmed air into the air conduct; and
c. creating an air flow extending through the heating mechanism and the device's delivery conduit; and
d. triggering the heating mechanism to activate when the patient actuates the device; and
e. triggering the heating mechanism to deactivate when the patient ceases to actuate the device; and possibly
f. flavoring the resulting warmed air by having it pass through an elective flavoring chamber, which houses a replaceable aerosol flavoring compound.

9. A method of assisting a person to ease their anxiety by warming them from the inside by providing a controlled delivery of warm air into their respiratory tract; the method comprising:
a. use of a Metered-dose Inhaler, Dry-powder Inhaler, Nebulizing Inhaler, or the inhaler claimed in claim 7 of this invention to increase an individual's temperature; and
b. introducing a warmed air into the air conduct; and
c. creating an air flow extending through the heating mechanism and the device's delivery conduit; and
d. triggering the heating mechanism to activate when the patient actuates the device; and
e. triggering the heating mechanism to deactivate when the patient ceases to actuate the device; and possibly
f. flavoring the resulting warmed air by having it pass through an elective flavoring chamber, which houses a replaceable aerosol flavoring compound.
